SEQUENCE LISTING (15 sequences)

SEQ ID NO: 1 - DNA, 7885 bp, Homo sapiens
  CDS (46)..(2427)
gcgaaggcgacagctctaggggttggcaccggccccgagaggaggatgcgggtccggatagggctgacgctgctgctgtgtgcggtgctgctgagcttggcctcggcgtcctcggatgaagaaggcagccaggatgaatccttagattccaagactactttgacatcagatgagtcagtaaaggaccatactactgcaggcagagtagttgctggtcaaatatttcttgattcagaagaatctgaattagaatcctctattcaagaagaggaagacagcctcaagagccaagagggggaaagtgtcacagaagatatcagctttctagagtctccaaatccagaaaacaaggactatgaagagccaaagaaagtacggaaaccagctttgaccgccattgaaggcacagcacatggggagccctgccacttcccttttcttttcctagataaggagtatgatgaatgtacatcagatgggagggaagatggcagactgtggtgtgctacaacctatgactacaaagcagatgaaaagtggggcttttgtgaaactgaagaagaggctgctaagagacggcagatgcaggaagcagaaatgatgtatcaaactggaatgaaaatccttaatggaagcaataagaaaagccaaaaaagagaagcatatcggtatctccaaaaggcagcaagcatgaaccataccaaagccctggagagagtgtcatatgctcttttatttggtgattacttgccacagaatatccaggcagcgagagagatgtttgagaagctgactgaggaaggctctcccaagggacagactgctcttggctttctgtatgcctctggacttggtgttaattcaagtcaggcaaaggctcttgtatattatacatttggagctcttgggggcaatctaatagcccacatggttttgggttacagatactgggctggcatcggcgtcctccagagttgtgaatctgccctgactcactatcgtcttgttgccaatcatgttgctagtgatatctcgctaacaggaggctcagtagtacagagaatacggctgcctgatgaagtggaaaatccaggaatgaacagtggaatgctagaagaagatttgattcaatattaccagttcctagctgaaaaaggtgatgtacaagcacaggttggtcttggacaactgcacctgcacggagggcgtggagtagaacagaatcatcagagagcatttgactacttcaatttagcagcaaatgctggcaattcacatgccatggcctttttgggaaagatgtattcggaaggaagtgacattgtacctcagagtaatgagacagctctccactactttaagaaagctgctgacatgggcaacccagttggacagagtgggcttggaatggcctacctctatgggagaggagttcaagttaattatgatctagcccttaagtatttccagaaagctgctgaacaaggctgggtggatgggcagctacagcttggttccatgtactataatggcattggagtcaagagagattataaacaggccttgaagtattttaatttagcttctcagggaggccatatcttggctttctataacctagctcagatgcatgccagtggcaccggcgtgatgcgatcatgtcacactgcagtggagttgtttaagaatgtatgtgaacgaggccgttggtctgaaaggcttatgactgcctataacagctataaagatggcgattacaatgctgcagtgatccagtacctcctcctggctgaacagggctatgaagtggcacaaagcaatgcagcctttattcttgatcagagagaagcaagcattgtaggtgagaatgaaacttatcccagagctttgctacattggaacagggccgcctctcaaggctatactgtggctagaattaagctcggagactaccatttctatgggtttggcaccgatgtagattatgaaactgcatttattcattaccgtctggcttctgagcagcaacacagtgcacaagctatgtttaatctgggatatatgcatgagaaaggactgggcattaaacaggatattcaccttgcgaaacgtttttatgacatggcagctgaagccagcccagatgcacaagttccagtcttcctagccctctgcaaattgggcgtcgtctatttcttgcagtacatacgggaaacaaacattcgagatatgttcacccaacttgatatggaccagcttttgggacctgagtgggacctttacctcatgaccatcattgcgctgctgttgggaacagtcatagcttacaggcaaaggcagcaccaagacatgcctgcacccaggcctccagggccacggccagctccaccccagcaggaggggccaccagagcagcagccaccacagtaataggcactgggtccagccttgatcagtgacagcgaaggaagttatctgctgggaacacttgcatttgatttaggaccttggatcagtggtcacctcccagaagaggcacggcacaaggaagcattgaattcctaaagctgcttagaatctgatgcctttattttcagggataagtaactcttacctaaactgagctgaatgtttgtttcagtgccatatggagtaacaactttcagtggcttttttttttcttttctggaaacatatgtgagacactcagagtaatgtctactgtatccagctatctttctttggatccttttggtcattatttcagtgtgcataagttcttaatgtcaaccatctttaaggtattgtgcatcgacactaaaaactgatcagtgttaaaaaggaaaacccagttgcaagtttaaacgtgttcgaaagtctgaaaatagaacttgccttttaagttaaaaaaaaaaaaaaagctatcttgaaaatgttttggaactgcgataactgagaaacttcttaccagtccacatgcaattaaacatattcagcatatttgttattttaaaagggagggttgggaggtttcttattggtgattgtcacacggtataccatactcctctccttcaaagaatgaaaggccttgttaaggagttttttgtgagctttacttctttggaatggaatatacttatgcaaaaccttgtgaactgactccttgcactaacgcgagtttgccccacctactctgtaatttgcttgtttgttttgaatataacagagccttgatccagaagccagaggatggactaagtgggagaaattagaaaacaaaacgaactctggttggggtactacgatcacagacacagacatacttttcctaaagttgaagcatttgttcccaggatttattttactttgcatttctttttgcacaaagaacacatcaccttcctgaattctttaaatatgaaatatcattgccagggtatggcttacagtgactactattatcaatactaaaactcagagaatcaaagatggattaaactcagtggttgatgaaagccaaaacctgtttgtactgttctatactattcaggtatctttttatttctgatagttttatattataatagaaagccagccactgcttagctatcatagtcaccattttctcactgttaacattaggaaaatcaaggctactatgcttcaggattgtctggttaaatagtatgggaaaaaaactgaagagtttcaacataattacacacgtgaaataattacagcttaaactgaatttgtatttcattttattgtcagatggtggtgttcaccagcctgtatcttgtctgagactgcattcgtatctgagcaggttttctatgcctactgatgtcagtatgtttatactaaccttcatgcttttttcccagaatccctcatctgccagaaaacttgaaaagtttattgcttgtagagttgtactgctttgatttttgaagttggggtagtagttagaactagatttaactagtctataatgaacatgaaggcttttatatatgaagttgtatacctttttgtgtttagagaattatgggaaacctggtaagcaaaactttcctcccagataattgcttccaaattcgaagagttagtcaccaagagagccatatgtatgaaagcgtatctgtgaaaggtaggaaacttaccccccctaagtgtaatgttgctttaggcaactcttgtaaatagtgagacttgtttggtctcttacatgtagagatttgagtgcagttggtacagtactttggtgtctccaccactgtcccttctccccgcttcaaaataagtgtaatccacggtagcagccacacttccttcagaaggaactgttataatttatttaaaagttgaaaaaccacccaagatgactaccaactttcactttttttcttctgccatccaccctcattttccctttagcaagatttttatatctaactttccttccctccattgagtacgtgctttgagaaaacatttcttaaaacagtgtgtgccacctaaggctggatgggaaagtgcagtcttgttgttcatataaaaaacacacttcttattagtttacccacttgcctttttctattgttaatgttctgaatttccttttcttggcttgtttctacttcattttaaccctgggtcacttgctgccagcagtttgtgaatggtgtctttcaaataacttagttcttatggcttcacttaaagactgtctcaaaaatactttgctctcttcttcttttttgttcatgggacatggtacctaagcaaataggagttgggtttggtttttctcctaaaataatgctcaatacttacctaatcaaatggcatccatttgaataaaatgacaataactaaagctagttaatgtcagtgacattaaactaactccaggattcaggagttttaatgttagaatttagatttaacagatagagtgtggcttcatttgtccatggtagcccatctctcctaagaccttttctagtctgtcttcctgccttcgaacttgatgacagtaaaaccctgtttagtattctcttgtgcatttggtttgttggttagccgactgtcttgaaactattcattttgcttctagttttattttacagaggtagcattggtgggtttttttttttttttctgtctctgtgtttgaagtttcagtttctgttttctaggtaaggcttatttttgattagcagtcaatggcaaagaaaaagtaaatcaaagatgacttcttttcaaaatgtatggcccttttattgcacttttaactcagatgaatttataaattattaatcttgatactaaggatttgttacttttttgcatattaggttaatttttaccttacatgtgagagtcttaccactaagccattctgtctctgtactgttgggaagttttggaaacccctgccagtgatctggtgatgatctgatgatttatttaaagagccgttgatgcctccaggaaacttaagtattttattaatatatatataggaatttttttttattttgctttgtctttctctcccttcttttatcctcatgttcattcttcaaaccagtgttttggaagtatgcatgcaggcctataaatgaaaaacacaattctttatgtgtatagcatgtgtattaatgtctaactacatacgcaaaaacttcctttacagaggttcggactaacatttcacatgcacatttcaaaacaagatgtgtcatgaaaacagcccctttacctgccaagacaagcagggctatatttcagtgacagctggatattttgtttctgaaagtgaatctcataatatatatatgtattacacattattatgactagaagtatgtaagaaatgatcagaacaaaagaaaatttctattttcatgcaaatatttttcatcagtcatcactctcaaatataaattaaaatataacactcctgaatgcctgaggcacgatctggattttaaatgtgtggtattcattgaaaagaagctctccacccacttggtatttcaagaaaatttaaaacgatcccaaggaaagatgatttgtatgttaaagtgactgcacaagtaaaagtccaatgttgtgtgcatgaaaaggattccttggttatgtgcagggaatcatctcacatgctgtttttcctatttggtttgagaaacaggctgacactattctctttgattagaaaataaactcataaaactcataatgttgatataatcaagatgttaaccactataaatatgtagaagaggaagttttaaatagaccttaagctggcattgtgaaggaacaccatggtagactctttttggtaatggtattttgtatttaatgaaatgcagtataaaggttggtgaagtgtaataataattgtgtaaacaaatcctgtttaatagaagagatgtacagaatcgttttggtactgtatcttgaaacttgtgaaataaagattccacttttggttatcctgtatgctgtaatataccacaaccaagcaccctttccagacagactttttttaagctgaatgaatccaattttttaatgttttttggaaattcagaagcttctgaaaacattcacttgtggcaatttgaatttatctttcattttaaactcctgaaattcagatttttacaagtccaatattgccctagggagaacatgaatttgctaagaaatgttatcttttaaatctctgatatctttgtcttgaagcagccttgatatgtagtaagcgtgattcactttagcctgattataatattatttatctaaagtttgtttatgcattgccttgtcccaggaattttttaagaggacttgcagagacacgtaccacacagtaacatttagactaaatatgctctgagtaaaggagaaatgaaaaaatattaaatcaagagtgaacatgtacacaaagtgcaattggaagtgggctacaaatttagcccccagcttcccagcaggcaactcaaagaggtaactgaggtaaaatgttccagctcagaagcattggatcttggataaaaagcctacatgatgcaaactgtggcaactgagatgtcagatctcaagatctcaaattgtacttgtgggagcacagtcagtgaccccagatgaccttgactgacctaaaagttgtgggggaagtcggatgtcagagccttaacaccagcaggtgaccatccaacctggggcaatgcctgcctgttcaccacttagcctctttctggcaagtcattagaatgtcctccatcttcattggctgcaacttgatgagctacagcctctttcctaacttcctttatgatgctagtttaggttggttataccagcttggaagtatgcttagattaagttacagcagatacacaaattagatgcaagtaaaaaaaatcagaatttctgtagtagaaactacgaaaaataaaaaggaaagtttttactttttgggtatttttttacgaataagaaaaagtgagcgttaatcagttcaaaaggaggtactgctgtgtaatgggctttgtacgttccttctcatgtcacttacgtcactacttcgccatcaaattgaacaagcttttaattagatcctgaaaattcactatgctagtagtttattggtagtattatattttgagtagaactctgattttccctagaggccaaattctttttatctgggttaatttcttttaaacataacaatgttaatgctgaattgtatattaaatcccatttctaaaaaccacacaattttttctcatgtaagttgagtggaatgtggttagttaactgaatttggaatgttcatataaataatttgttgctgctc

SEQ ID NO: 2 - protein, 794 aa, Homo sapiens
MRVRIGLTLLLCAVLLSLASASSDEEGSQDESLDSKTTLTSDESVKDHTTAGRVVAGQIFLDSEESELESSIQEEEDSLKSQEGESVTEDISFLESPNPENKDYEEPKKVRKPALTAIEGTAHGEPCHFPFLFLDKEYDECTSDGREDGRLWCATTYDYKADEKWGFCETEEEAAKRRQMQEAEMMYQTGMKILNGSNKKSQKREAYRYLQKAASMNHTKALERVSYALLFGDYLPQNIQAAREMFEKLTEEGSPKGQTALGFLYASGLGVNSSQAKALVYYTFGALGGNLIAHMVLGYRYWAGIGVLQSCESALTHYRLVANHVASDISLTGGSVVQRIRLPDEVENPGMNSGMLEEDLIQYYQFLAEKGDVQAQVGLGQLHLHGGRGVEQNHQRAFDYFNLAANAGNSHAMAFLGKMYSEGSDIVPQSNETALHYFKKAADMGNPVGQSGLGMAYLYGRGVQVNYDLALKYFQKAAEQGWVDGQLQLGSMYYNGIGVKRDYKQALKYFNLASQGGHILAFYNLAQMHASGTGVMRSCHTAVELFKNVCERGRWSERLMTAYNSYKDGDYNAAVIQYLLLAEQGYEVAQSNAAFILDQREASIVGENETYPRALLHWNRAASQGYTVARIKLGDYHFYGFGTDVDYETAFIHYRLASEQQHSAQAMFNLGYMHEKGLGIKQDIHLAKRFYDMAAEASPDAQVPVFLALCKLGVVYFLQYIRETNIRDMFTQLDMDQLLGPEWDLYLMTIIALLLGTVIAYRQRQHQDMPAPRPPGPRPAPPQQEGPPEQQPPQ

SEQ ID NO: 3 - protein, 833 aa, Saccharomyces cerevisiae
MITLLLYLCVICNAIVLIRADSIADPWPEARHLLNTIAKSRDPMKEAAMEPNADEFVGFYVPMDYSPRNEEKNYQSIWQNEITDSQRHIYELLVQSSEQFNNSEATYTLSQIHLWSQYNFPHNMTLAHKYLEKFNDLTHFTNHSAIFDLAVMYATGGCASGNDQTVIPQDSAKALLYYQRAAQLGNLKAKQVLAYKYYSGFNVPRNFHKSLVLYRDIAEQLRKSYSRDEWDIVFPYWESYNVRISDFESGLLGKGLNSVPSSTVRKRTTRPDIGSPFIAQVNGVQMTLQIEPMGRFAFNGNDGNINGDEDDEDASERRIIRIYYAALNDYKGTYSQSRNCERAKNLLELTYKEFQPHVDNLDPLQVFYYVRCLQLLGHMYFTGEGSSKPNIHMAEEILTTSLEISRRAQGPIGRACIDLGLINQYITNNISQAISYYMKAMKTQANNGIVEFQLSKLATSFPEEKIGDPFNLMETAYLNGFIPAIYEFAVMIESGMNSKSSVENTAYLFKTFVDKNEAIMAPKLRTAFAALINDRSEVALWAYSQLAEQGYETAQVSAAYLMYQLPYEFEDPPRTTDQRKTLAISYYTRAFKQGNIDAGVVAGDIYFQMQNYSKAMALYQGAALKYSIQAIWNLGYMHEHGLGVNRDFHLAKRYYDQVSEHDHRFYLASKLSVLKLHLKSWLTWITREKVNYWKPSSPLNPNEDTQHSKTSWYKQLTKILQRMRHKEDSDKAAEDSHKHRTVVQNGANHRGDDQEEASEILGFQMEDLVTMGCILGIFLLSILMSTLAARRGWNVRFNGAQLNANGNRQQEQQQQQQAQGPPGWDFNVQIFAI

SEQ ID NO: 4 - DNA, 21 bp, Artificial
  DNA/RNA binding molecule
cuugauauggaccagcuuutt

SEQ ID NO: 5 - DNA, 21 bp, Artificial
  DNA/RNA binding molecule
aaagcugguccauaucaagtt

SEQ ID NO: 6 - DNA, 21 bp, Artificial
  DNA/RNA binding molecule
ggcuacguccaggagcgcatt

SEQ ID NO: 7 - DNA, 21 bp, Artificial
  DNA/RNA binding molecule
ugcgcuccuggacguagcctt

SEQ ID NO: 8 - DNA, 23 bp, Artificial
  DNA/RNA binding molecule
gguguucuuugggcaacugagtt

SEQ ID NO: 9 - DNA, 23 bp, Artificial
  DNA/RNA binding molecule
cucaguugcccaaagaacacctt

SEQ ID NO: 10 - DNA, 17 bp, Artificial
  synthetic DNA
ggctgaacagggctatg

SEQ ID NO: 11 - DNA, 30 bp, Artificial
  synthetic DNA
ccgctcgagttactgtggtggctgctgctc

SEQ ID NO: 12 - DNA, 20 bp, Artificial
  synthetic DNA
agctggtgtttggctttgag

SEQ ID NO: 13 - DNA, 20 bp, Artificial
  synthetic DNA
gggtggcccctgatccgcag

SEQ ID NO: 14 - DNA, 23 bp, Artificial
  synthetic DNA
aggtgaaggtcggagtcaacgga

SEQ ID NO: 15 - DNA, 24 bp, Artificial
  synthetic DNA
agtccttccacgataccaaagttg

(19) United States
(12) Patent Application Publication
(10) Pub. No.: US 2007/0238677 A1
(43) Pub. Date: Oct. 11, 2007

(54) Pharmaceutical Composition Containing Hshrd3

(75) Inventors: Toshihiro Nakajima, Yokohama-shi (JP); Tetsuya Amano, Kawasaki-shi (JP); Satoshi Yamasaki, Yokohama-shi (JP); Naoko Yagishita, Yokohama-shi (JP); Takeshi Sasaki, Yokohama-shi (JP); Yukihiro Kato, Yamato-shi (JP); Lei Zhang, Yokohama-shi (JP)

Correspondence Address: DICKSTEIN SHAPIRO LLP, 1177 AVENUE OF THE AMERICAS (6TH AVENUE), NEW YORK, NY, 10036-2714, US

(73) Assignee: LOCOMOGENE, INC., Yokohama-shi (JP)

(21) Appl. No.: 10/592,918

(22) PCT Filed: Mar. 16, 2005
(86) PCT No.: PCT/JP05/05311

§ 371 Date: Feb. 2, 2007

(30) Foreign Application Priority Data
Mar. 17, 2004 (JP) 2004-076931
Oct. 28, 2004 (JP) 2004-314364

(51) Int. Cl. A61K 31/7088; A61K 45/00; A61K 48/00; A61P 19/02; A61P 29/00; A61P 35/00; C12N 15/09; C12N 5/00

(52) U.S. Cl. 514/44; 435/375

(57) ABSTRACT

The present invention provides a pharmaceutical composition containing a substance which inhibits the proliferation of synovial tissue or synovial cells and the interleukin-6 production. It also provides a pharmaceutical composition for inhibiting the proliferation of synovial tissue or synovial cells and the interleukin-6 production, which is useful for diagnosing and treating at least one selected from the group consisting of rheumatoid arthritis, fibrosis, arthritis, cancers, and cerebral neural diseases. In addition, it provides a method for inhibiting the proliferation of synovial cells and the interleukin-6 production by inhibiting the expression of hsHRD3 in synovial cells.

### TECHNICAL FIELD

The present invention relates to a pharmaceutical composition containing a human Hrd3 ortholog (hsHRD3) which forms a complex with Synoviolin, and in particular relates to a pharmaceutical composition for diagnosing and treating rheumatoid arthritis.

### BACKGROUND ART

Rheumatoid arthritis (hereinafter referred to as RA) is a systemic chronic inflammatory disease wherein hyperplasia is seen in the synovial tissue of joints. The inventors identified the synoviolin gene as an essential gene to hyperplasia of the synovial tissue (WO 02/052007).

Synoviolin is a membrane protein which is present in the RA patient-derived synovial cells and which encodes E3 ubiquitin ligase having a RING finger motif. This motif plays an important role in ubiquitination of proteins. In fact, it was proved to have an auto-ubiquitination activity and to cause the ubiquitination of proteins that are essential to collagen synthesis called “P4HA1” (WO 02/052007). In addition, it was recently discovered that Synoviolin is involved in the onset of fibrosis, cancers, and cerebral neural diseases (Genes Dev. 2003 Vol. 17: p. 2436-49).

Synoviolin is highly preserved in species from yeast to humans and detailed analyses have been published using the budding yeast. Hrd1p (HMG-COA Reductase Degradation 1), which is a budding yeast ortholog of Synoviolin, is a gene relating to cholesterol reductive enzymatic degradation and it is known to form a functional complex with Hrd3p (HMG-CoA Reductase Degradation 3), which is involved in the degradation of aberrant proteins in the endoplasmic reticulum (J.B.C. 2000, Vol. 151, p. 69-82). However, functions regarding Hrd3p have not yet been clarified.

Interleukin-6, as well as interleukin-1 and TNF-α, is called an “inflammatory cytokine” that causes a variety of inflammatory reactions. The cells in immune systems generally produce it, but it is also produced from the cells inducing diseases due to hyperplasia such as rheumatoid synovial cells, leukemia, and myeloma. This indicates that Interleukin-6 is essential to proliferation. The diseases that interleukin-6 is associated with include RA, multiple myeloma, Castleman's disease, Crohn's disease, systemic juvenile idiopathic arthritis, systemic lupus erythematosus, osteoporosis, and the like. Interleukin-6 may bind to the interleukin-6 receptor expressed on the cell surface, but it also binds to the receptor freed from the cell surface, to be linked to the cells not expressing the receptor that induces inflammatory reactions. Interleukin-6's inflammatory actions include differentiation of B-cells into the antibody reproducing cells, increased production of C-reactive proteins in the liver, induction of platelets in bone marrow, induction of immune cells into the inflammation sites, contribution to a resistance to apoptosis in the white blood cells, VEGF-mediated vascular invasion, etc. Recently, an anti-interleukin-6 antibody that inhibits binding of interleukin-6 to the receptors was prepared, exhibiting the effects in RA, myeloma, Crohn's disease, and the like.

### DISCLOSURE OF THE INVENTION

The purpose of the present invention is to provide a pharmaceutical composition containing a substance which inhibits hyperplasia of synovial cells and interleukin-6 production, as well as a method of inhibiting proliferation of synovial cells by inhibiting hsHRD3.

The inventors earnestly explored an attempt to solve the aforementioned problems. The fact that in the budding yeast hrd3 destruction line, Hrd1p proteins became destabilized and decreased, while the substrate was physiologically stabilized and increased suggested that human Hrd3p ortholog is also essential for hyperplasia of synovial cells and interleukin-6 production, as well as Synoviolin. We presumed that use of hsHRD3 is effective for inhibiting new inflammatory reactions and for developing a diagnostic method and a treatment method for RA, fibrosis, arthritis, cancers, and cerebral neural diseases, which achieved the present invention.

The present invention is as described below.
(1) A pharmaceutical composition containing a substance which inhibits the proliferation of synovial cells.

As a substance which inhibits the proliferation of synovial cells, an expression-inhibitory substance for Synoviolin may be given, for example. The expression-inhibitory substance for Synoviolin includes a substance which inhibits the expression of a gene encoding hsHRD3, preferably a siRNA (small interfering RNA) or shRNA (short hairpin RNA) against the gene encoding hsHRD3.

Specifically, the gene encoding hsHRD3 comprises a DNA selected from the following (a) or (b):

(a) A DNA consisting of the nucleotide sequence as shown in SEQ ID NO: 1;

(b) A DNA which hybridizes to a DNA complementary to a DNA consisting of the nucleotide sequence as shown in SEQ ID NO: 1 under stringent conditions, and encodes a protein having hsHRD3 activity.

Further, the siRNA may target a portion of the nucleotide sequence as shown in SEQ ID NO: 1.

The pharmaceutical composition of the present invention can be used for diagnosing of treating at least one disease selected from the group consisting of RA, fibrosis, arthritis, cancers, and cerebral neural diseases.
(2) A method of inhibiting the proliferation of synovial cells, characterizing inhibiting the expression of hsHRD3 in synovial cells.
(3) A method of inducing apoptosis in synovial cells, cancer cells, leukemia, and malignant tumors, characterizing inhibiting the expression of hsHRD3 in the synovial cells.
(4) A method of inhibiting production of collagen in the synovial cells, pulmonary fibrosis, and hepatocirrhosis, which is characterized by inhibiting the expression of hsHRD3 in synovial cells.
(5) A method of inhibiting interleukin-6 production in at least one cell selected from the group consisting of synovial cells, cancer cells, leukemia cells, osteosarcoma cells, malignant tumor cells, immune system cells, and osteoclasts, characterizing inhibiting the expression of hsHRD3 in synovial cells.
(6) A pharmaceutical composition containing a substance which inhibits interleukin-6 production.

As a substance which inhibits interleukin-6 production, an expression-inhibitory substance for synoviolin may be given, for example. The expression-inhibitory substance for synoviolin includes a substance which inhibits the expression of a gene encoding hsHRD3, preferably a siRNA or shRNA against the gene encoding hsHRD3.

Specifically, the gene encoding hsHRD3 comprises a DNA selected from the following (a) or (b):

(a) A DNA consisting of the nucleotide sequence as shown in SEQ ID NO: 1;

(b) A DNA which hybridizes to a DNA complementary to a DNA consisting of the nucleotide sequence as shown in SEQ ID NO: 1 under stringent conditions, and encodes a protein having hsHRD3 activity.

Further, the siRNA may target a portion of the nucleotide sequence as shown in SEQ ID NO: 1.

The pharmaceutical composition of the present invention can be used for diagnosing or treating at least one disease selected from the group consisting of RA, multiple myeloma, Castleman's disease, Crohn's disease, systemic juvenile idiopathic arthritis, systemic lupus erythematosus, and osteoporosis. In addition, the pharmaceutical composition of the present invention can inhibit inflammatory reactions.

In the aforementioned methods (2) through (5), the expression of hsHRD3 in synovial cells may be inhibited, for example, by inhibiting binding between hsHRD3 and Synoviolin.

### BRIEF DESCRIPTION OF THE DRAWINGS

FIG. 1 is a diagram showing the domain structures of Hrd3p and SEL1L/hsHRD3.

FIG. 2 is a diagram showing that the expression of SEIL/hsHRD3 by siRNA is inhibited.

FIG. 3 is a diagram showing that the proliferation activity of the synovial cells is inhibited by inhibition of SEIL/hsHRD3 expression.

FIG. 4 is a diagram showing that apoptosis of synovial cells is induced by the inhibition of SEIL/hsHRD3 expression.

FIG. 5 is a diagram showing apoptosis into synovial cells is induced by the inhibition of SEIL/hsHRD3 expression.

FIG. 6 is a diagram showing that Synoviolin protein in the synovial cells is reduced by the inhibition of SEIL/hsHRD3 expression.

FIG. 7 is a diagram showing that collagen production in the synovial cells is inhibited by the inhibition of SEIL/hsHRD3 expression.

FIG. 8 is a diagram showing that a complex is formed between SEIL/hsHRD3 and Synoviolin.

FIG. 9 is a diagram showing co-localization of SEIL/hsHRD3 and Synoviolin in the endoplasmic reticulum.

FIG. 10 is a diagram showing that production of interleukin-6 in the synovial cells is inhibited by the inhibition of SEL1L/hsHRD3 expression.

FIG. 11 is a diagram showing that the expression of both proteins is inhibited by the inhibition of SEL1L/hsHRD3 expression and Synoviolin.

FIG. 12A is a diagram showing that SEL1L/hsHRD3 is unstable in the absence of Synoviolin.

FIG. 12B is a diagram showing that SEL1L/hsHRD3 is unstable in the absence of Synoviolin.

### BEST MODE FOR CARRYING OUT THE INVENTION

The present invention is explained in detail below.

The present invention relates to a pharmaceutical composition containing a substance which inhibits hyperplasia of synovial cells and interleukin-6 production by inhibiting the expression of hsHRD3, which is effective for diagnosing and treating diseases such as rheumatoid arthritis.

Synoviolin is highly preserved in species from yeast to humans and detailed analyses have been published using the budding yeast. Hrd1p, which is a budding yeast ortholog of Synoviolin is known to form a functional complex with Hrd3p that is involved in the degradation of aberrant proteins in the endoplasmic reticulum. It was reported that in the hrd3 destruction line of budding yeast, Hrd1p proteins became destabilized and reduced, while the substrate was physiologically stabilized and increased. Human Hrd3p ortholog (hsHRD3) is also essential for hyperplasia of synovial cells so that it is effective for developing a new diagnosis and treatment method for arthritis, as well as Synoviolin.

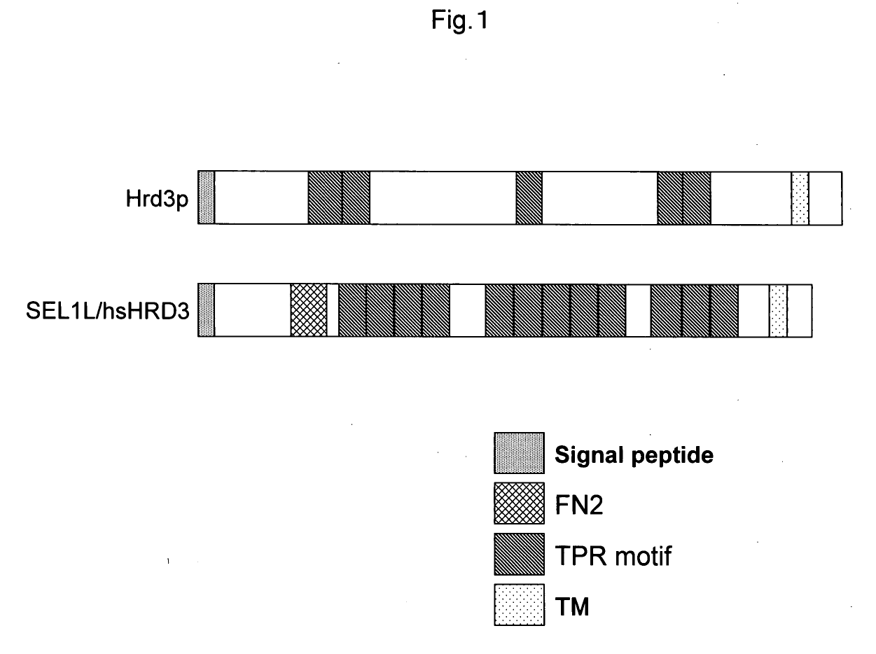

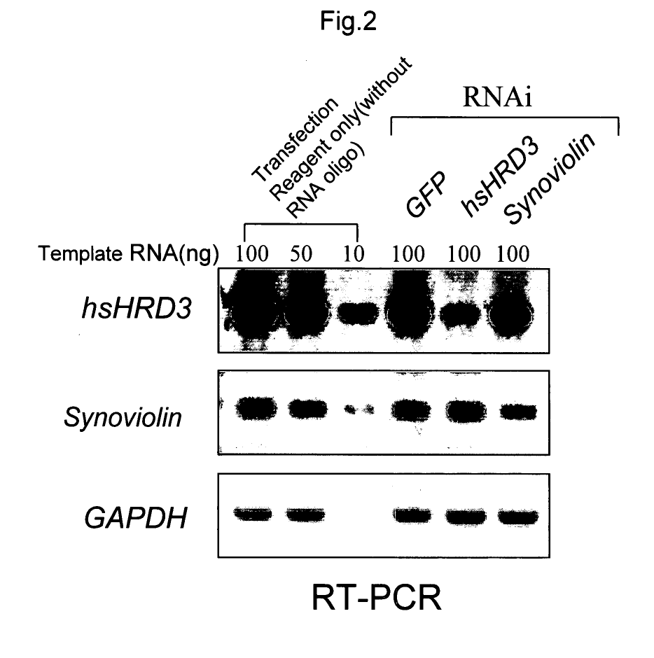

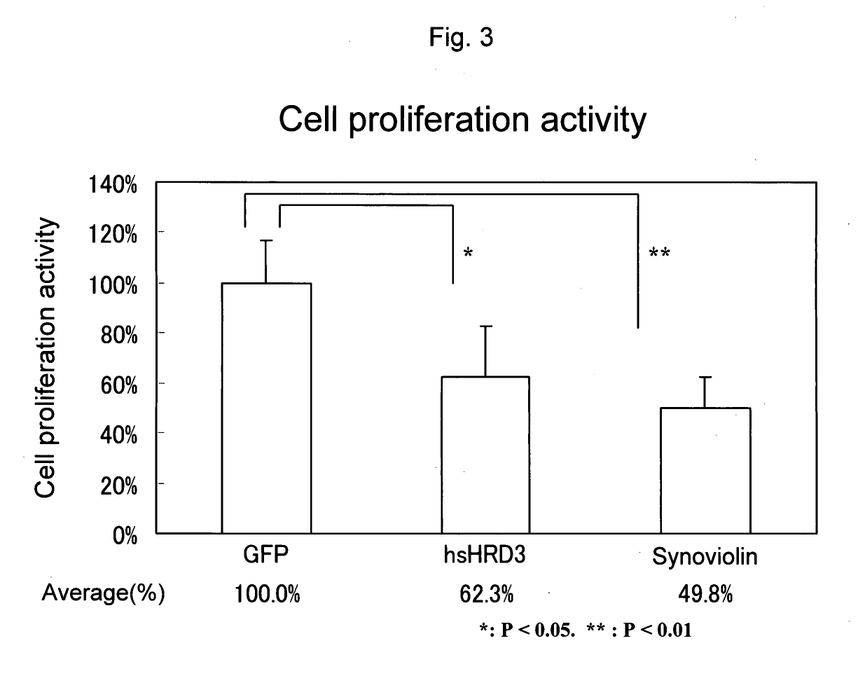

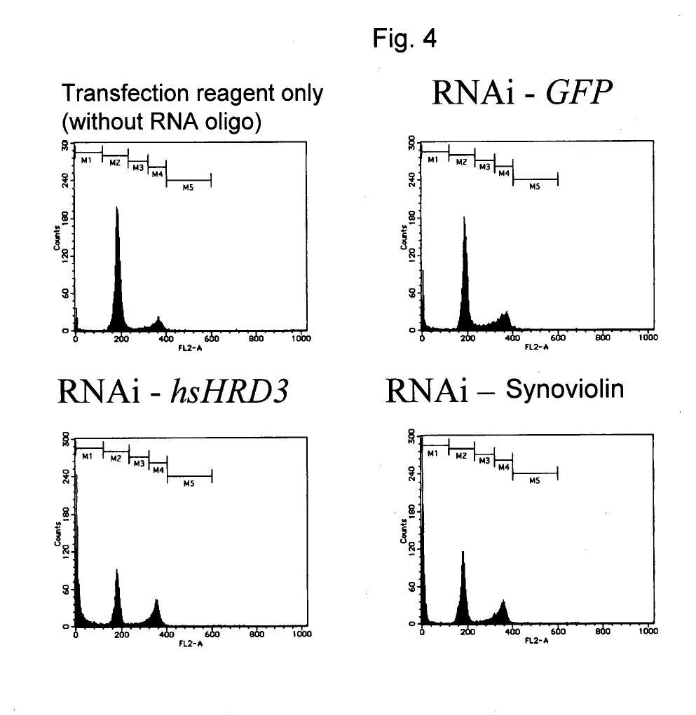

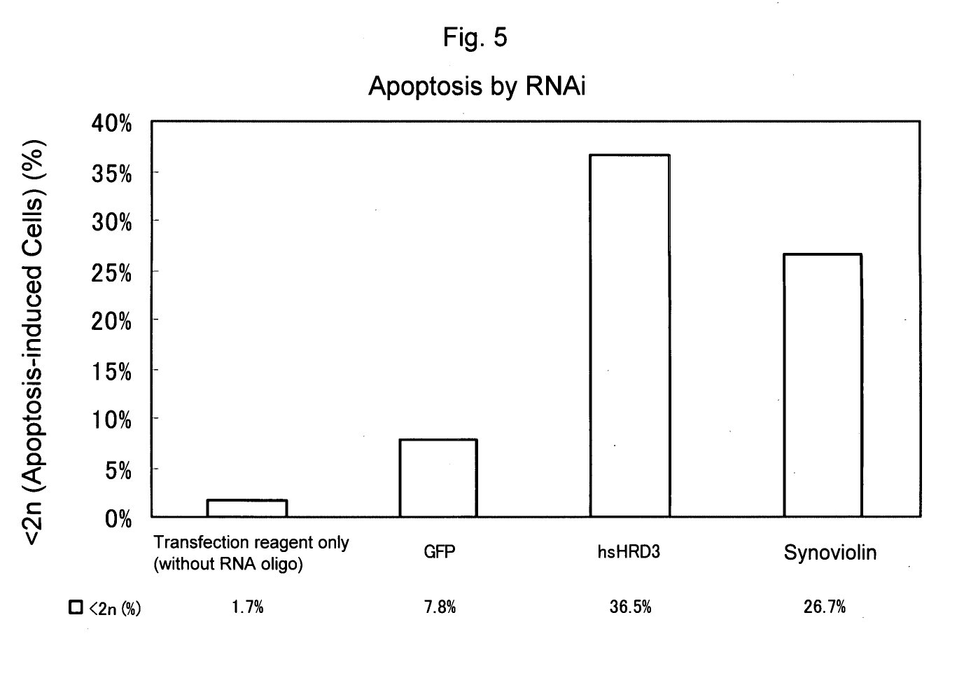

In the present invention, using the amino acid sequence of the budding yeast Hrd3p, homology was searched and as a result, an existing gene called SEL1L was discovered. Homology of the amino acid sequence between Hrd3p and SEL1L was found to be 30%, and similarity was found to be 45%. Homology and similarity were not so high, but a specific repeating structure and a transmembrane domain were found to be preserved. Thus, SEL1L was determined to be an ortholog of Hrd3p (FIG. 1). Next, we confirmed that when the synovial cells were treated with a double stranded RNA (siRNA), the expression of hsHRD3 could be inhibited (FIG. 2). Under such conditions, cellular proliferation activity in the synovial cells was significantly reduced (FIG. 3). Also, approximately 30% of cells underwent apoptosis (FIGS. 4 and 5).

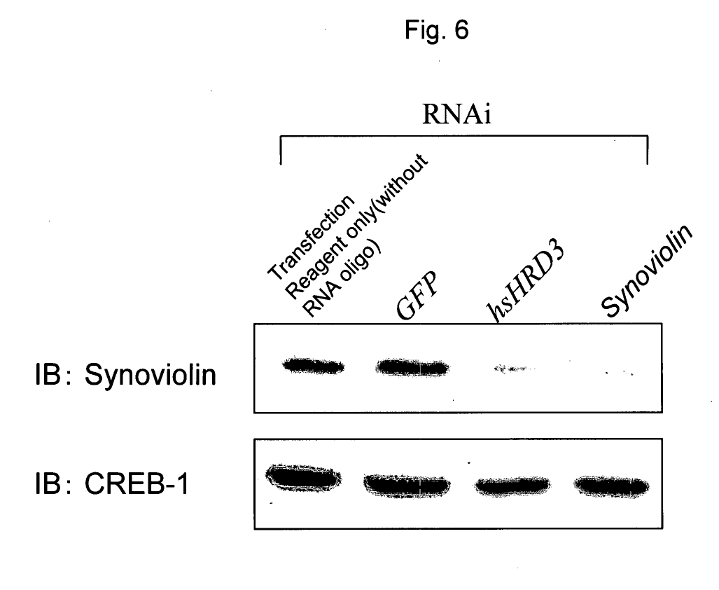

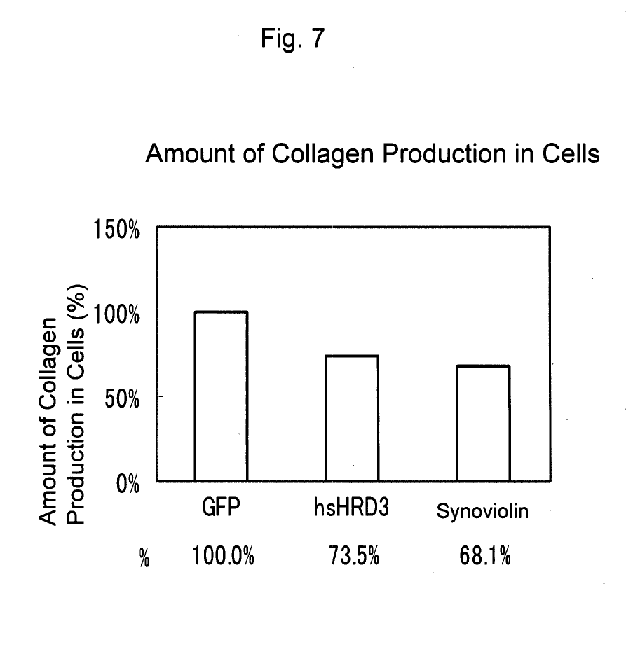

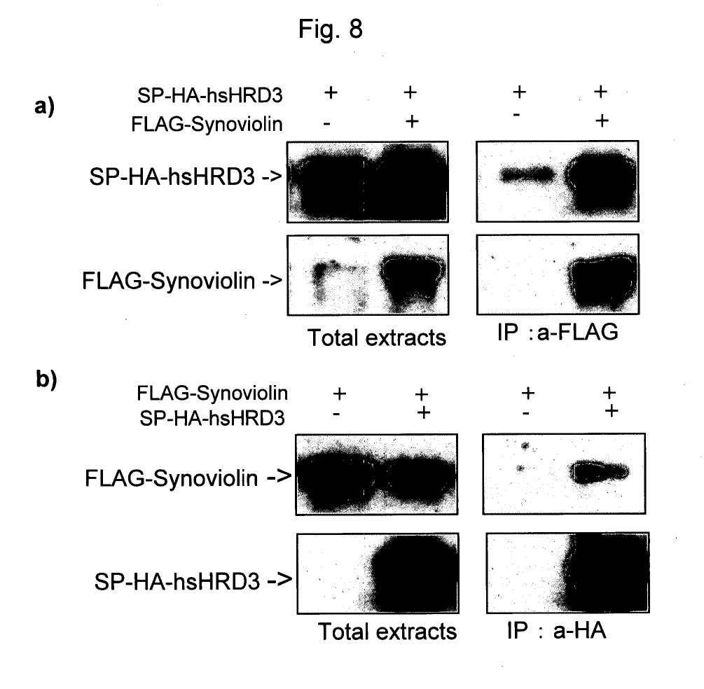

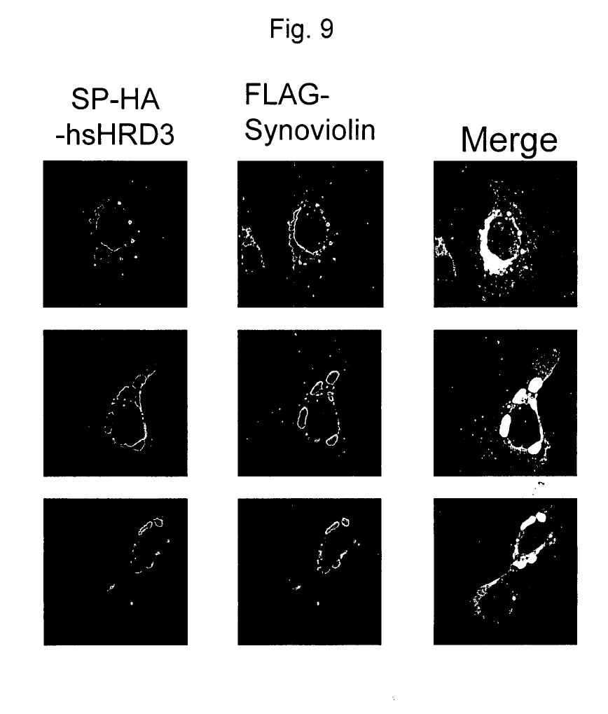

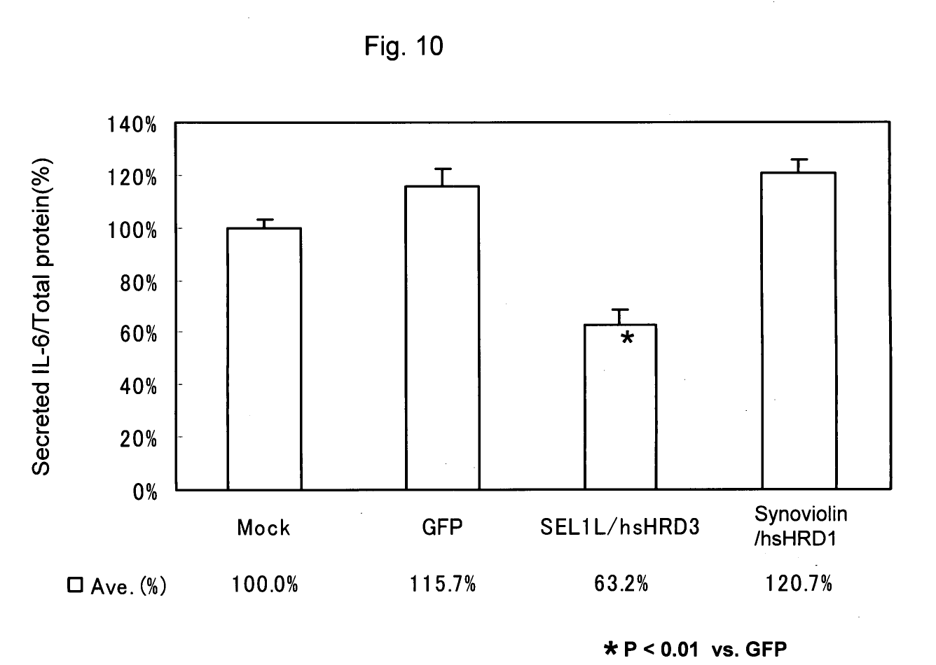

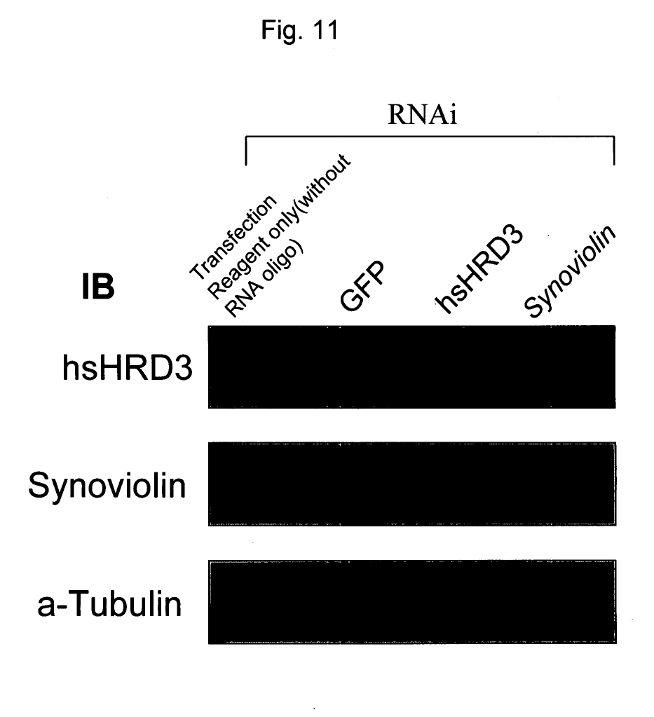

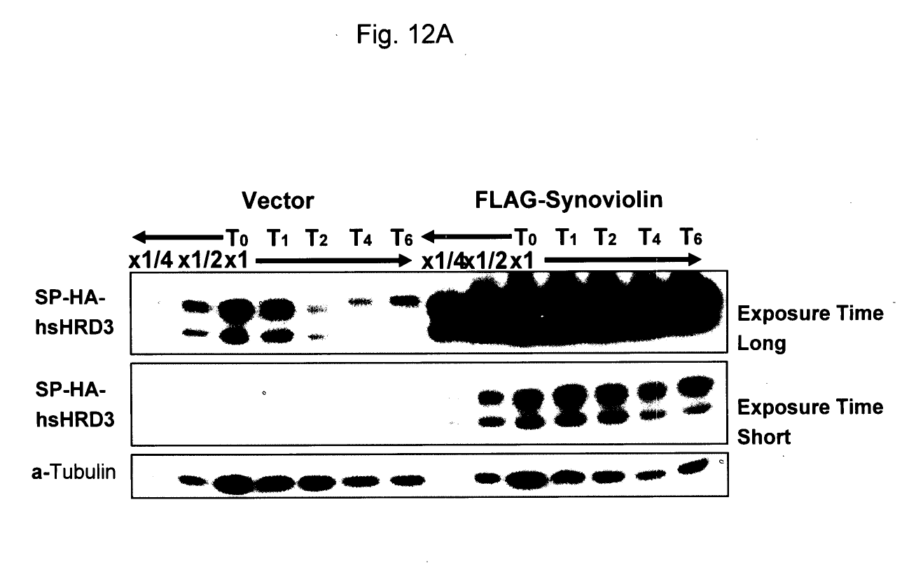

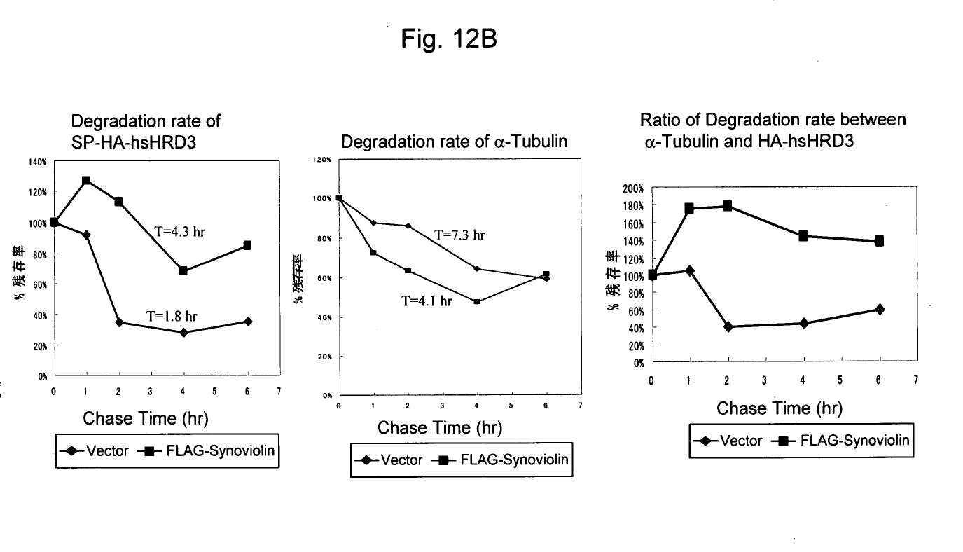

In the budding yeast, Hrd3p is essential for the stabilization of Hrd1p. Synoviolin protein was found to be reduced significantly under inhibition of hsHRD3, which detected by Western blotting (FIG. 6). When the expression of Synoviolin was inhibited, the amounts of collagen production decreased. When the amount of collagen in the cells was measured, it was also reduced when compared to that of control (FIG. 7). Moreover, hsHRD3 formed a complex with Synoviolin in the cells (FIG. 8), and both were found to be localized in the endoplasmic reticulum (FIG. 9). In addition, interleukin-6, playing an important role in proliferation of the synovial cells, decreased to 63.2% (FIG. 10). Also, if Synoviolin protein is not present in the cells, hsHRD3 decreased significantly (FIG. 11), and became very unstable (FIGS. 12A and 12B).

The aforementioned results suggested that the approach targeting hsHRD3 is effective for developing a new diagnosis and treatment method for diseases such as arthritis including RA, fibrosis, cancer, and cerebral neural diseases. In particular, it is useful for developing drugs based on the mechanism of action that the expression and function of Synoviolin are controlled via control of the expression and functions of SEL1L/hsHRD3.

1. Inhibition of Hyperplasia in Synovial Cells

In the present invention, “synovial cells” implies a grouping of a series of cells showing hyperplasia at the joint site of RA patients and includes synovial membrane tissue.

In the present invention, “hsHRD3” forms a functional complex when binding to Hrd1p, which is Synoviolin in yeast, and is a human ortholog of the protein called “Hrd3p” that is involved in the degradation of aberrant protein in the endoplasmic reticulum. Synoviolin has been highly preserved in species from yeast to humans, and in particular, a detailed analysis has been conducted using the budding yeast. The homology of an ortholog (Hrd3p) of the budding yeast for amino acids was 30% and the similarity was 45%. A gene preserving the specific repeating structure and a transmembrane domain was discovered and called “SEL1L”, which was later named “hsHRD3”. This hsHRD3 consists of the nucleotide sequence as shown in SEQ ID NO: 1 and the nucleotide sequence that was substantially identical to the sequence. The substantially identical nucleotide sequence is the nucleotide sequence obtained by hybridizing DNA consisting of SEQ ID NO: 1 with DNA consisting of its complementary nucleotide sequence under stringent conditions, with that encoded protein having the hsHRD3 activity. The “hsHRD3 activity” is an activity that degrades aberrant protein in the endoplasmic reticulum. This DNA encoding hsHRD3 can be obtained by a method known by those in the art wherein a probe is prepared using an appropriate fragment that can be obtained by known hybridization, such as colony hybridization, plaque hybridization, Southern blotting, etc., from the cDNA and genome libraries. The stringent conditions in said hybridization are as follows: for example, a salt concentration during washing in the hybridization is 100 to 500 mM, and preferably 150 to 300 mM, and a temperature ranging from 50° C. to 70° C., and preferably from 55° C. to 65° C.

The amino acid sequence of hsHRD3 is shown as SEQ ID NO: 2 and the amino acid sequence of Hrd3p is shown as SEQ ID NO: 3.

If the expression of this hsHRD3 is inhibited, the proliferation activity of the synovial cells is extremely inhibited. The synovial cells are the cells that become regular joint constitutional elements and that produce a synovial fluid, filling up the inside layer of the joint cavity.

In order to inhibit the expression of the synoviolin gene, a method of inhibiting the expression of hsHRD3 is employed. In order to inhibit the expression of hsHRD3, a phenomenon called RNAi can be used. However, a site-directed mutagenesis system using genetic engineering, anti-sense nucleotide and ribozyme can be employed.

RNAi is a phenomenon in which dsRNA (double-strand RNA) binds to a target gene, specifically and selectively, to cut the target gene such that the expression can be inhibited more efficiently. For example, when dsRNA is transfected into cells, the expression of a gene with the identical sequence as RNA is inhibited (knock down).

In order to induce RNAi, for example, siRNA or shRNA for the synoviolin gene is designed and synthesized, and then the product is reacted. Alternately, inhibiting the expression of the gene encoding hsHRD3 can inhibit the expression of Synoviolin.

The standards for designing siRNA are as follows.
(a) A domain downstream by 100 nucleotides from the initiation codon of the gene encoding Synoviolin is selected.
(b) From the selected domain, a sequence consisting of sequential 15 to 30 nucleotides, starting with AA and preferably 19 nucleotides is searched with the sequence having a GC content of 30% to 70%, and preferably a content of 35% to 45% is selected.

Specifically, those having the following nucleotide sequence can be used as siRNA:

| | |
|---|---|
| Sense chain: | |
| CUUGAUAUGGACCAGCUUUTT | (SEQ ID NO: 4) |
| Anti-sense chain: | |
| AAAGCUGGUCCAUAUCAAGTT | (SEQ ID NO: 5) |

To transfected the siRNA into the synovial cells, siRNA synthesized in vitro is linked to plasmid DNA or a method of annealing double-stranded RNA can be employed.

Thus, the synovial cells are treated with siRNA and the expression of the hsHRD3 is inhibited.

In order to achieve the RNAi effect, shRNA can be used. shRNA is called a short hairpin RNA. It is an RNA molecule having a stem loop structure that forms a complementary chain between a portion of the area of a single strand and another area.

shRNA can be designed to form a section that is a stem loop structure. For example, if a sequence of a certain region is sequence A, and the complementary chain to the sequence A is sequence B, sequence A+spacer+sequence B in this order form a single RNA chain with a total of 45 to 60 nucleotides is designed. Sequence A is the sequence of a part of the region in the target hsHRD3 gene (SEQ ID NO: 1), but the target region is not particularly limited and an arbitrary region can be selected. The length of sequence A is 19 to 25 nucleotides and preferably 19 to 21 nucleotides.

Proliferation of the synovial cells may be measured by the following method. An appropriate amount of alamarBlue is added into culture medium. Using the excitation wavelength of 540 nm for several hours, a fluorescent intensity of 590 nm is measured.

Moreover, in order to inhibit the expression of the synoviolin gene, or gene encoding hsHRD3, a site-directed mutagenesis system can be used. This site-directed mutagenesis system is known to those in the art and a commercial kit, for example GeneTailor™ Site-Directed Mutagenesis System (Invitrogen Corp.) or TAKARA Site-Directed Mutagenesis System (Mutan-K, Mutan-Super Express Km, etc. (Takara Bio Corp.)), are available for use.

According to the present invention, the provided is a method of inhibiting the expression of Synoviolin by inhibiting the formation of a complex formed by binding hsHRD3 to Synoviolin, which is localized in the endoplasmic reticulum.

Once hsHRD3 binds to Synoviolin to form a complex, the expression of Synoviolin are increases. In this case, the complex of hsHDR3 and Synoviolin is localized in the endoplasmic reticulum. The protein in the middle of biosynthesis in the lumen of the endoplasmic reticulum is unstable so that it is exposed to various physiochemical stresses (e.g. ischemia, hypoxia, heat shock, amino acid starvation, gene mutation, etc.). These stresses are called endoplasmic reticulum stress (ER stress), which increases the frequency of occurrence for proteins having an abnormally folded structure (an unfolded protein) in the endoplasmic reticulum. Since the defective or damaged proteins, having an unusual tertiary structure due to disability to form a conformation, are not transported from the endoplasmic reticulum into the Golgi apparatus, defective proteins are accumulated in the endoplasmic reticulum. The cells decompose defective proteins due to the stress response mechanism specific to the endoplasmic reticulum called UPR (unfolded protein response) and ERAD (endoplasmic reticulum-associated degradation) in order to protect the endoplasmic reticulum from being stressed due to an accumulation of defective proteins. Thus, the quality of the endoplasmic reticulum is controlled to maintain the homeostasis of cellular functions. In the budding yeast hrd3 destruction line, Hrd1p proteins became unstable and reduced, whereas the substrate is observed to be physiologically stabilized and increased. Therefore, in humans, hsHRD3 forming a complex with Synoviolin is presumed to be related to this quality control mechanism.

That is, once the expression of hsHRD3 is inhibited, hsHRD3 bound to Synoviolin is reduced, resulting in an inhibition of the expression of Synoviolin.

Also, if the expression of Synoviolin increases, ERAD is hyperfunctioned so that sensitivity to apoptosis due to ER stress is reduced. In contrast, if the expression of Synoviolin is suppressed, sensitivity to apoptosis increases. Therefore, once the expression of hsHRD3 is suppressed, the function of Synoviolin decreases and as a result, apoptosis is increased.

On the other hand, in terms of collagen, through the ubiquitination of protein that is essential to collagen synthesis called “P4HA1”, the quality as an enzyme is maintained so that Synoviolin plays an essential role in the collagen synthesis. Once the expression of Synoviolin is inhibited, the enzyme activity of P4HA1 decreases, resulting in reduced collagen synthesis. Thus, once the expression of hsHRD3 is suppressed, the function of Synoviolin decreases and as a result, collagen synthesis decreases.

Therefore, hsHRD3 is essential to hyperplasia in the synovial tissue as well as Synoviolin. Therefore, if the expression of hsHRD3 is inhibited, the following changes will take place: proliferation of the synovial cells is inhibited, induction of apoptosis of the synovial cells, cancer cells, leukemia and malignant tumor is induced, and the production of collagen is inhibited in the synovial cells, pulmonary fibrosis, and hepatocirrhosis. As a result, it is possible to develop a new diagnostic method and therapeutic method for RA, fibrosis, arthritis, cancer, and cerebral neural diseases.

The aforementioned inhibitor of Synoviolin expression, which inhibits proliferation of the synovial cells, is also a substance that inhibits interleukin-6 production.

Interleukin-6 is a typical cytokine, having multi-functions that play an important role in broad immune responses, hematopoiesis, inflammatory reactions, cellular proliferation and differentiation in the nervous systems, or expression of these functions as well as proliferation and differentiation of B-lymphocytes. Its actions include the induction of platelets in the bone marrow, induction of immune cells into the inflammation sites, contribution to a resistance to the apoptosis of white blood cells, VEGF-mediated vascular induction, and differentiation of B-cells into antibody producing cells, and increased C—reactive protein production in the liver. Interleukin-6 is generally produced from the cells in the immune system, but it is also produced from the cells causing hyperplasia diseases such as RA synovial cells, leukemia, and myeloma, indicating that it is essential to such proliferation. The diseases that interleukin-6 is associated with include RA, multiple myeloma, Castleman's disease, Crohn's disease, systemic juvenile idiopathic arthritis, systemic lupus erythematosus, osteoporosis, and the like. In chronic inflammatory hyperplasia, interleukin-6 is known to play an important role in the formation of lesions.

It is also known that the abnormal expression of the gene encoding interleukin-6 induces the onset of autoimmune diseases such as RA, the onset of multiple myeloma caused by a cancerous transformation of the blood cells, and plasmacytoma such as leukemia. For example, it was found that interleukin-6 markedly increased in the joint fluid of the RA patients, and that the growth factor in the plasmacytoma/multiple myeloma is interleukin-6 itself. Interleukin-6 reacts with the myelogenous leukemia cells to inhibit proliferation and to induce differentiation of microphage.

Therefore, if the interleukin-6 production is inhibited in the synovial cells, cancer cells, leukemia cells, osteosarcoma cells, malignant tumor cells, immune cells, and osteoclasts, the onsets of autoimmune diseases such as RA, multiple myeloma caused by cancerous transformation of the blood cells, and leukemia can be inhibited.

In order to inhibit interleukin-6 production, a synoviolin expression inhibitory substance that inhibits proliferation of the synovial cells can be used. Specifically, substances for inhibiting the expression of the gene encoding hsHRD3, such as siRNA or shRNA for the gene encoding hsHRD3, can be used.

2. Pharmaceutical Composition

(1) Pharmaceutical Composition Comprising a Substance which Inhibits a Proliferation of the Synovial Cells

Indications of the pharmaceutical composition of the present invention include diseases attributed to cellular hyperplasia such as RA, fibrosis, arthritis, cancers, and cerebral neural diseases. When the pharmaceutical composition of the present invention is applied to these diseases, said diseases can be present singly, or associations of multiple diseases are included within the subjects of application.

If the pharmaceutical compositions of the present invention are used as cancer treatment drugs, types of cancers are not restricted. For example, target cancers include brain tumors, tongue cancer, pharyngeal cancer, lung cancer, breast cancer, esophageal cancer, gastric cancer, pancreatic cancer, gall bladder cancer, biliary tract carcinoma, duodenum cancer, colon cancer, liver cancer, uterine cancer, ovarian cancer, prostate cancer, renal cancer, bladder cancer, rhabdomyosarcoma, fibrosarcoma, osteosarcoma, chondrosarcoma, skin cancer, various kinds of leukemias (e.g., acute myelogenous leukemia, acute lymphatic leukemia, chronic myelogenous leukemia, chronic lymphatic leukemia, adult T-cell leukemia, malignant lymphoma), etc.

The aforementioned cancers include the primary lesion or metastasis. Other diseases can be associated.

Cerebral neural diseases include Alzheimer's disease, Parkinson's disease, and polyglutamine disease.

(2) Pharmaceutical Composition Comprising a Substance which Inhibits Interleukin-6 Production

Indications of the pharmaceutical composition of the present invention include RA, multiple myeloma, Castleman's disease, Crohn's disease, systemic juvenile idiopathic arthritis, systemic lupus erythematosus, osteoporosis, and the like.

In addition, interleukin-6 is also a cytokine that causes numerous symptoms associated with inflammations (pain, fever, etc.). Therefore, the pharmaceutical composition of the present invention can inhibit inflammatory reactions as well. The inflammatory reactions include localized tissue reactions in a living body caused by stimulants such as infections, external injuries, burn, or allergens and also systemic phenomena associated with local reactions are included. Specifically, they are referred to as the 5 characteristics of inflammations, which are redness, fever, pain, swelling and functional disorders. These represent macroscopic characteristics of acute inflammations, but the phenomena are localized vascular changes, which include dilation of blood vessels, hyperfunction of permeability, and infiltration of white blood cells.

The mode of administration of the pharmaceutical composition of the present invention comprising a substance as an active ingredient for inhibiting hyperplasia in the synovial tissue and inhibiting interleukin-6 production, can be either an oral or parenteral route. In the case of oral administration, syrup or an appropriate drug form can be used. In the case of parenteral administration, via pulmonary administration types (e.g., using a nebulizer, etc.), via nasal administration types, percutaneous injection types (e.g., ointments, cream agents), and injection types are available. In the case of injection types, agents can be administered systemically or locally, via various drip fusions such as intravenous injection, intramuscular injection, intraperitoneal injection and subcutaneous injection.

If the pharmaceutical composition of the present invention is used as a gene therapy, in addition to direct administration by the injection of the composition, a method of administering a vector incorporating a nucleic acid is available. As the aforementioned vectors, adenoviral vector, adeno-associated viral vector, herpes viral vector, vaccinia viral vector, retroviral vector, lentiviral vector, and the like are available. Use of these viral vectors makes administration more efficient.

A pharmaceutical composition of the present invention can be introduced into a phospholipids vesicle, such as a liposome, and the vesicle can be administered. A vesicle retaining a pharmaceutical composition of the invention is transfected to a specific cell by the lipofection method. The cells obtained are then administered systemically intravenously or intra-arterially. They can be administered locally, for example to the brain. In order to introduce the pharmaceutical composition of the present invention into the target tissues and organs, commercial gene transfection kits (e.g. Adeno Express: Clontech Corp.) can be used.

The pharmaceutical composition of the present invention can be formulated by a conventional method and can contain pharmaceutically acceptable carriers and additives. Such carriers and additives are as follows: water, organic solvents that are pharmaceutically acceptable, collagen, polyvinyl alcohol, polyvinylpyrrolidone, carboxyvinyl polymer, sodium carboxymethylcellulose, sodium polyacrylate, sodium alginate, water-soluble dextran, sodium carboxymethyl starch, pectin, methylcellulose, ethyl cellulose, xanthan gum, gum Arabic, casein, agar-agar, polyethylene glycol, diglycerin, glycerin, propylene glycol, Vaseline, paraffin, stearyl alcohol, stearic acid, human serum albumin, mannitol, sorbitol, lactose, and surfactants that are acceptable as pharmaceutical additives.

The aforementioned additives can be selected singly or in combination according to the types of formulas as treatment drugs of this invention. For example, when used as an injection formula, a purified substance inhibiting hyperplasia in the synovial tissue is dissolved in a solvent (e.g., saline, buffer solution, glucose solution, etc.) and then mixed with Tween 80, Tween 20, gelatin, human serum albumin, etc. Alternatively, it can be freeze-dried so that it can be dissolved before use. Sugar alcohols and other sugars, such as mannitol and glucose, are available as a freeze-dry forming agent.

Doses of the pharmaceutical composition of the present invention vary with age, sex, symptoms, administration routes, frequency of administration, and types of formulas. A method of administration is appropriately selected based on patients' ages and symptoms. An effective daily dose ranges from 0.1 μg to 100 mg per kg bodyweight and preferably from 1 to 10 μg. However, the aforementioned treatment agent is not limited by these dosages. For example, if an adeno virus is administered, a single daily dose is approximately 10⁶ to 10¹³ and is administered with an interval of 1 week to 8 weeks. However, the pharmaceutical composition of the present invention is not limited by these dosages. A dosage when mixing siRNA ranges from 0.01 to 10 μg/ml, and preferably from 0.1 to 1 μg/ml.

This invention will be described in detail with reference to the examples. However, this invention will not be limited by these examples.

### EXAMPLE 1

Searching for Homology Using the Budding Yeast Hrd3p

A homology search was executed using an amino acid sequence from the budding yeast Hrd3p/Y1r207wp.

As a result, a protein corresponding to the amino acid sequence encoded by the nucleotide sequence as shown in SEQ ID NO: 1, which is a human ortholog of the yeast Hrd3p was identified and the SEL1L gene was discovered. The amino acid sequence of Hrd3p is shown in SEQ ID NO: 3. The homology of the amino acid sequence between Hrd3p and SEL1L was 30% and similarity was 45%, both of which showed low values. But, the specific repeating structure and the transmembrane domain were preserved. Therefore, SEL1L was identified as an ortholog of Hrd3p (FIG. 1).

### EXAMPLE 2

Investigation of the Expression Inhibition of SEL1L/hsHRD3

(1) The RA synovial cells were transfected by double-stranded RNA (siRNA) against each gene, and after 96 hours the cells were recovered. After extracting the RNA, the amount of expression for each gene was quantitatively determined by RT-PCR.

That is, on the day before transfection, the synovial cells isolated from RA patients were seeded on a 6 cm dish at 1×10⁴ cells per 6 cm dish. Each sample of three kinds of oligos for RNAi, and one without an RNA oligo (a negative control), was scattered (one dish per each sample, total number of dishes: 4). A 3 ml of antibiotic-free DMEM (Dulbecco's Modified Eagle's Medium, Sigma D6046), supplemented with 10% FBS (fetal bovine serum) was used. After 24 hours, the culture dish was washed once with 3 ml of DMEM, not containing a serum or antibiotic substance, and then 1.6 ml of the same DMEM was added.

Subsequently, a transfection reagent was added. The transfection reagent was prepared as follows.

For RNAi targeted for GFP, hsHRD3 and Synoviolin, RNA oligos as indicated in the following sequences (SEQ ID NO: 4˜9) were dissolved in TE to achieve a final concentration of 100 μM.

| | |
|---|---|
| siRNA sense chain targeted hsHRD3: | |
| CUUGAUAUGGACCAGCUUUTT | (SEQ ID NO: 4) |
| siRNA anti-sense chain targeted hsHRD3: | |
| AAAGCUGGUCCAUAUCAAGTT | (SEQ ID NO: 4) |
| siRNA sense chain targeted GFP: | |
| GGCUACGUCCAGGAGCGCATT | (SEQ ID NO: 6) |
| siRNA anti-sense chain targeted GFP: | |
| UGCGCUCCUGGACGUAGCCTT | (SEQ ID NO: 7) |
| siRNA sense chain targeted Synoviolin: | |
| GGUGUUCUUUGGGCAACUGAGTT | (SEQ ID NO: 8) |

siRNA anti-sense chain targeted Synoviolin:

| | |
|---|---|
| CUCAGUUGCCCAAAGAACACCTT | (SEQ ID NO: 8) |

Sense chain and anti-sense chain of RNA oligos were mixed to achieve 20 μM relative to each gene. After being thermally denatured at 90° C. for 2 minutes, both oligos were annealed by slowly cooling at 37° C. for 1 hour. After annealing, 10 μl of 20 μM RNA oligo and 350 μl of OptiMEM were mixed to prepare Solution A. Subsequently, 8 μl of Oligofectamine™ Reagent (Invitrogen, Cat. No. 12252-011) was mixed with 32 μl of OptiMEM to prepare Solution B. After incubating Solution A and Solution B for 5 minutes, both solutions were combined and the mixture was further incubated for 15 minutes. The entire solution mixture of 400 μl was added to each dish in which the culture had been replaced. After 4 hours, 200 μl of FBS was added.

After 96 hours from the addition of the transfection reagent, total RNA was extracted from the cells by phenol extraction method and used for RT-PCR. For RT-PCR, SUPERSCRIPT™ One-step RT-PCT 100 Reactions (Invitrogen Cat. No. 10928-042) was used. That is, 2×RXN mixture 50 μl, RT/Platinum 2 μl, DEPC water 28 μl and each set of the following primer 3.2 μM solution for amplification 10 μl×2 were mixed to a total of 100 μl, and then poured into each PCR tube in portions (10 μl each portion). The PCR reaction was initiated by adding 1 μl of RNA as a RT-PCR template.

Oligomer (5′->3′) for amplification of hsHRD3:

| | |
|---|---|
| GGCTGAACAGGGCTATG | (SEQ ID NO: 10) |

Oligomer (3′->5′) for amplification of hsHRD3:

| | |
|---|---|
| CCGCTCGAGTTACTGTGGTGGCTGCTGCTC | (SEQ ID NO: 11) |

Oligomer (5′->3′) for amplification or Synoviolin:

| | |
|---|---|
| AGCTGGTGTTTGGCTTTGAG | (SEQ ID NO: 12) |

Oligomer (3′->5′) for amplification of Synoviolin:

| | |
|---|---|
| GGGTGGCCCCTGATCCGCAG | (SEQ ID NO: 13) |

Oligomer (5′->3′) for amplification of hGAPDH:

| | |
|---|---|
| AGGTGAAGGTCGGAGTCAACGGA | (SEQ ID NO: 14) |

Oligomer (3′->5′) for amplification of hGAPDH:

| | |
|---|---|
| AGTCCTTCCACGATACCAAAGTTG | (SEQ ID NO: 15) |

100, 50 and 10 ng for RNA oligo-free and 100 ng for others was used as a template. The cycle consisted of the following reactions: cDNA elongation carried out once at 50° C. for 30 minutes, and 94° C. for 2 minutes, followed by PCR amplification reaction at 94° C. for 30 seconds, 50° C. for 30 seconds, and 72° C. for one minute, which was repeated 30 times, and a final elongation reaction carried out at 72° C. for 5 minutes, and then the reaction mixture was stored at 4° C. A 2 μl of a 6× sample buffer was added to the PCR reaction solution, and the entire amount was treated with 0.8% agarose by electrophoresis at 100 V for 30 minutes, and the PCR products were observed with a UV illuminator.

The results showed that the expression of SEIL/hsHRD3 was found to be inhibited by siRNA (FIG. 2). As shown in FIG. 2, the amounts of PCR products were found to be reduced by RNAi of hsHRD3 to the same level as that in the case of 10 ng of oligo-free (a negative control), indicating that the expression level of mRNA of hsHRD3 was inhibited to 10% or less. Since the mRNA of Synoviolin was at the same level as that of 100 ng oligo-free and GFP RNAi, it was found that the expression inhibition of hsHRD3 had no effect on the transcription of Synoviolin.

(2) The RA synovial cells were transfected by double-strand RNA (siRNA) against each gene and after 48 hours, alamarBlue™ was added. After an additional 48 hours, the cellular proliferation activity was measured.

That is, on the day before transfection, the synovial cells isolated from RA patients were seeded on each 96-well plate at 160 cells. The culture medium used was a 100 μl of antibiotic-free DMEM supplemented with 10% FBS. After 24 hours, the cells were washed once with a 100 μl of serum- or antibiotic-free DMEM, and then 80 μl of the the same DMEM was added. Subsequently, the transfection reagent prepared by the same method as in Example 2 (1) (20 μl each) was added to each well in which the culture medium had been replaced. After an additional 4 hours, 10 μl of FBS was added. At 48 hours after the addition of the transfection reagent, 10 μl of alamarBlue™ was added to each well. After incubation at 37° C. for 48 hours, a fluorescent intensity of 590 nm when excited at 560 nm was measured.

The results demonstrated that to suppress the expression of SEIL/hsHRD3, the proliferation activity in the synovial cells was inhibited about 60% (FIG. 3).

This implies that hsHRD3 is important in cell proliferation of the RA synovial cells as well as Synoviolin, and that the inhibition of its expression induces a reduction in cell proliferation.

(3) The RA synovial cells were transfected by double-stranded RNA (siRNA) for each gene and after 120 hours the cells were recovered. After staining the recovered cells with propidium iodide, the DNA amount was measured by FACS.

That is, on the day before transfection, the synovial cells isolated from RA patients were seeded on a 6 cm dish at 1×10⁴ cells per dish. Each sample of three kinds of oligos for RNAi and one without an RNA oligo (a negative control) was scattered (one dish per sample, total number of dishes: 4). A 3 ml of antibiotic-free DMEM, supplemented with 10% FBS, was used. After 24 hours, the culture dish was washed once with 3 ml of serum- and antibiotic-free DMEM, and then 1.6 ml of the same DMEM was added. A total of 400 μl of transfection reagent prepared by the same method as in Example 2 (1) was added to each dish in which the culture medium had been replaced. After an additional 4 hours, 200 μl of FBS was added.

After 120 hours from the addition of a transfection reagent, all cells were recovered and solubilized in 0.5 ml of PBS (−)/0.2% TritonX-100, and then cell clumps were removed through a nylon mesh. 1 ml of 50 μg/ml RNase/PBS (−) and 1 ml of 100 μg/ml propidium iodide/PBS (−) were added, mixed, and then stored in ice cold water. The level of fluorescence of each cell was measured using a FACSCalibur (Becton Dickinson) and analyzed using CellQuest.

As a result, as shown in FIG. 4, a group of cells having a DNA content of 2n or less, wherein apoptosis was presumed to be induced, has increased to 30% or greater by RNAi of hsHRD3. This ratio was as high as the ratio of RNAi for Synoviolin (FIG. 5). This implies that hsHRD3 is an essential gene for the proliferation of synovial cells, as in the case of Synoviolin, demonstrating that the inhibition of its expression induces a high frequency of apoptosis.

### EXAMPLE 3

(1) Detection of Synoviolin Using Western Blotting in the Inhibition of the Expression of SEL1L/hsHRD3

The RA synovial cells were transfected by double-stranded RNA (siRNA) against each gene and after 48 hours the cells were recovered. After total extraction, each protein was detected by Western blotting.

That is, on the day before transfection, the synovial cells isolated from RA patients were seeded on a 10 cm dish at 9×10⁴ cells per dish. Each sample of three kinds of oligos for RNAi and one without RNA oligo (a negative control) was scattered (one dish per sample, total number of dishes: 4). A 10 ml of antibiotic-free DMEM (Dulbecco's Modified Eagle's Medium, Sigma D6046), supplemented with 10% FBS (fetal bovine serum), was used. After 24 hours, the culture dish was washed once with 10 ml of serum- or antibiotic-free DMEM and then 9 ml of the same DMEM was added. Three times greater amount of transfection reagent (1.2 ml), prepared by the same method as in Example 2 (1), and was added to each dish wherein the culture medium has been replaced. After an additional 4 hours, 1 ml of FBS was added.

After 48 hours from the addition of a transfection reagent, all cells were recovered and re-suspended in 50 μl of extraction buffer IV (50 mM Tris-HCl pH 7.5, 2 mM EDTA, 0.1% Triton X-100, 1% NP-40, 500 mM NaCl, 1 mM phenylmethylsulfonyl fluoride (PMSF), 0.1% Aprotinin, 0.5 μg/ml Pepstatin A, and 1 μg/ml Leupeptin). After leaving in ice water for 30 minutes, the mixture was separated by centrifugation at 14000 rpm, 4° C. for 30 minutes. A 1 μl of supernatant was used for the measurement of protein concentrations using a Bio-Rad DC Protein Assay Reagent (BIO-RAD, Cat. No. 500-0116), and 15 μl of 4× SDS buffer was added to the remaining 45 μl of the supernatant and the mixture was heated at 100° C. for 5 minutes. The cell extracted solution in an equivalent of 10 μg was separated by electrophoresis using 2 sheets of 7.5% acrylamide gel. After blotting on a nitrocellulose membrane (OPTITRAN BA-S 85, Reinforced NC, Schleicher & Schuell, Cat. No. 10 439196), the membrane was blocked with 5% skim milk for 30 minutes.

As a primary antibody, 1000 times diluted anti-synoviolin monoclonal antibody (10 Da) or anti-CREB-1 antibody (Santa Cruze, Cat. No. sc-58) was incubated for 30 minutes. As a secondary antibody of the anti-synoviolin monoclonal antibody, 2000 times diluted HRP-linked anti-mouse IgG (Amersham Biosciences, Cat. No. NA931V), and 3000 times diluted HRP-linked anti-rabbit IgG (Amersham Biosciences, Cat. No. NA931V) were used as an anti-CREB-1 antibody, and incubated for 30 minutes. For detection, a Home-made ECL was used (44 μl of 90 mM coumaric acid, 100 μl of 250 mM leminor, and 6 μl of hydrogen peroxide solution were mixed in 20 ml of 100 mM Tris pH 8.5).

The results demonstrated that the Synoviolin protein was reduced significantly under the inhibition of SEL1L/hsHRD3 (FIG. 6). That is, it was clarified that the expression inhibition of hsHRD3 induces instability of Synoviolin protein.

(2) Investigation of Collagen Production under Inhibition of the Expression of Synoviolin

The RA synovial cells were transfected by double-stranded RNA (siRNA) against each gene and after 48 hours the cells were recovered. After preparation of the total extraction, the collagen level in the cells was measured.

That is, according to the same method as in Example 3 (1), the cells were treated by transfection and the cell extraction solution was prepared. A portion of extraction equivalent to 30 μg was adjusted to 100 μl using an extraction buffer IV, and the collagen level was measured using a SIRCOL Collagen Assay Kit (QBS Corp./Funakoshi Cat. No. S111).

The results showed that in the cells in which hsHRD3 was knocked out, the collagen level in the cells was reduced to about 70%, when compared to that in the control group (GFP) (FIG. 7).

That is, hsHRD3 accelerates collagen production via stabilization of Synoviolin protein. By inhibiting the expression of hsHRD3, the level of Synoviolin protein decreases, resulting in a reduction in the level of collagen production.

### EXAMPLE 4

Formation of a Complex between SEL1L/hsHRD3 and Synoviolin in the Cells

HEK293 cells were transfected with plasmids of SP-HA-hsHRD3B and FLAG-Synoviolin. After 48 hours the cells were recovered and a total extraction solution was prepared. Immunoprecipitation was carried out with anti-FLAG antibodies (a) or anti-HA antibodies (b), and Western blotting was performed using the respective antibodies.

That is, a plasmid (SP-HA-hsHRD3B) in which DNA was constructed, such that a HA-tag is inserted between the 26^{th} position and the 27^{th} position in the amino acid sequence indicated by SEQ ID NO: 1 immediately after the signal peptide (SP) of hsHRD3B is cloned in the pcDNA3-vecter.

8×10⁵ cells of HEK293 were seeded on four 10 cm dishes. After 24 hours, plasmids with the following four combinations (c) to (f) were transfected.

(c) 10 μg of SP-HA-hsHRD3B/pcDNA3 and 3 μg of pCAGGS-vector

(d) 10 μg of SP-HA-hsHRD3B/pcDNA3 and 3 μg of FLAG-Synoviolin/pCAGGS

(e) 10 μg of pcDNA3-vector and 3 μg of FLAG-Synoviolin/pCAGGS

(f) 10 μg of SP-HA-hsHRD3B/pcDNA3 and 3 μg of FLAG-Synoviolin/pCAGGS

At 48 hours after the transfection, the cells were recovered and resuspended in 200 μl of extraction buffer II (10 mM Tris-HCl pH 7.5, 150 mM NaCl, 0.5% NP-40, 10 mM MgCl₂10% glycerol, 5 mM EGTA, 20 mM NaF, 50 mM β-glycerophosphate, 1 mM Na₃VO₄, 10 mM NEM (N-ethylmaleimide), 1 mM PMSF, 1 mM DTT, 0.1% Aprotinin, 0.5 μg/ml Pepstatin A, 1 μg/ml Leupeptin). After incubating on ice for 30 minutes, the cell mixture was separated by centrifugation at 14000 rpm at 4° C. for 30 minutes. An extract equivalent to 100 μg of protein was prepared in an extraction buffer II at 1 ml. At the same time, a bovine serum albumin was added to achieve a final concentration of 0.5%.

Subsequently, 4.9 mg of anti-FLAG antibodies (M2, SIGMA, Cat. No. F3165) were added to the extract derived from transfections (c) and (d), or 2.4 mg of anti-HA antibodies (12CA5, Roche, Cat. No. 1 583 816) were added to the extract derived from transfections (e) and (f), and the mixture was incubated at 4° C. while being immersed overnight. On the following day, 60 μl of 50% slurry protein-G sepharose beads were added and the mixture was further incubated at 4° C. for 1 hour. The beads were rinsed twice with 0.5 ml extraction buffer II, twice with 0.5 ml extraction buffer II+150 mM NaCl (final concentration of 300 mM NaCl), and 30 μl of 2× SDS sample buffer was added and heated at 100° C. for 5 minutes in order to elute the proteins adsorbed. According to the same method in Example 3 (1), SDS-PAGE and Western blotting were performed, and the immunoprecipitated proteins were detected.

As a result, it was determined that SEL1L/hsHRD3 forms a complex with Synoviolin in the cells (FIG. 8).

(2) Co-Localization of SEL1L/hsHRD3 and Synoviolin in the Cells

HEK293 cells were transfected with the plasmids of SP-HA-hsHRD3B and FLAG-Synoviolin. After 24 hours, the cells were fixed and immunostaining was applied using anti-HA antibodies and anti-synoviolin monoclonal antibodies.

That is, 2000 cells of HEK293 were seeded in each chamber on the chamber slide. After 24 hours, transfection was performed with 0.15 μg of SP-HA-hsHRD3B/pcDNA3 and 0.05 μg of FLAG-Synoviolin. At 48 hours after the transfection, the cells were fixed with 4% paraformaldehyde for 30 minutes, and treated with 3% BSA/PBS (−) overnight for blocking. The cells were stained with anti-HA antibodies (3F10, Roche, Cat. No. 1 867 431) diluted with 0.3% BSA/PBS (−) and with anti-Synoviolin monoclonal antibodies (10 Da) that had been diluted by 100 times to achieve a final concentration of 1 ng/μl. The anti-HA antibodies were detected with anti-rat Ig FITC antibodies (DAKO, Cat. No. F0234) and anti-Synoviolin antibodies were detected with anti-mouse Ig TRITC antibodies (DAKO, Cat. No. R0270). Samples were observed and micrographs were taken using a confocal laser scanning microscopy LSM510 (Carl Zeiss Co., Ltd.). Images were analyzed by an LSM510-v3.0.

The results indicated that SEL1L/hsHRD3 and Synoviolin are co-localized in the endoplasmic reticulum (FIG. 9). In FIG. 9, the left column is a diagram of the localization of hsHRD3 (green), the center column is a diagram of the localization of Synoviolin (red), and the right column is a diagram when both are merged (yellow).

According to these results, it was determined that a complex of hsHRD3 and Synoviolin is formed in the endoplasmic reticulum.

### EXAMPLE 5

Investigation of the Interleukin-6 Production, Under the Inhibited Expression of SEL1L/hsHRD3

(1) The RA synovial cells were transfected by double-stranded RNA (siRNA) against each gene, and after 96 hours the culture medium was replaced with a new medium. Furthermore, after 24 hours the culture medium was recovered and the amount of interleukin-6 included in the medium was measured.

That is, on the day before transfection, the synovial cells isolated from RA patients were seeded on a 6 cm dish at 1×10⁴ cells per dish. Each sample of three kinds of oligos for RNAi and one without an oligo (a negative control) was scattered (one dish per sample, total number of dishes: 4). A 3 ml of antibiotic-free DMEM (Dulbecco's Modified Eagle's Medium, Sigma D6046), supplemented with 10% FBS (fetal bovine serum) was used. After 24 hours, the culture was washed once with 3 ml of serum- or antibiotic-free DMEM, and then 1.6 ml of the same DMEM was added. The entire 400 μl of the transfection reagent, prepared by the same method as in Example 2 (1), was added to each dish in which the medium had been replaced. After an additional 4 hours, 200 μl of FBS was added.

After 96 hours from the time when the transfection reagent was added, the culture medium was replaced with a new one. After culturing for 24 hours, the culture was recovered and centrifuged at 14000 rpm at 4° C. for 30 minutes. The amount of protein, interleukin-6 included, in the supernatant was measured with an ELISA kit (BIOSOURCE Immunoassay Kit for Human IL-6, Cat. # KHC0061). At the same time, the cells were recovered and dissolved in a 20 μL of extraction buffer III (10 mM Tris-HCl pH 7.5, 5 mM EDTA, 1% NONIDET P-40, 0.1% SDS, 200 mM NaCl 10 mM N-ethylmaleimide (NEM), 1 mM PMSF, 1 mM dithiothreitol, 0.1% Aprotinin, 0.5 μg/ml Pepstatin A, 1 μg/ml Leupeptin), and left on ice for 30 minutes. After centrifugation at 14000 rpm at 4° C. for 30 minutes, a 1 μl of supernatant was analyzed for the protein concentration, using a Bio-Rad DC Protein Assay Reagent (BIO-RAD, Cat. No. 500-0116), and the amount of total protein was calculated. The values obtained by dividing the amounts of interleukin-6 protein in the culture by the amounts of total protein were plotted on a graph (FIG. 10).

The results demonstrated that the production amount of interleukin-6 protein decreased to 63.2%, as compared to the control, due to the inhibition of the expression of SEL1L/hsHRD3 (FIG. 10). That is, it was found that SEL1L/hsHRD3 is an essential factor in the production of interleukin-6.

(2) A 15 μl of 4× SDS buffer was added to the 45 μl of total extraction solution of the cells prepared above in section (1), and the mixture was heated at 37° C. for 10 minutes. The cell-extracted solution in an equivalent of 10 μg was separated by electrophoresis using 7.5% acrylamide gel. After blotting on a nitrocellulose membrane (OPTITRAN BA-S 85, NC, Schleicher & Schuell, Cat. No. 10 439196), the membrane was blocked with 5% skim m REINFORCED ilk for 30 minutes.

Incubation was carried out for 30 minutes using 1000 times diluted anti-SEL1L/hsHRD3 peptide antibodies as the primary antibody. As a secondary antibody, 10000 times diluted HRP-bound anti-rabbit IgG (Amersham Biosciences, Cat. No. NA934V) was used for incubation, which was carried out for 30 minutes. For detection, an ECL plus Western Blotting Detection System (Amersham Biosciences, Cat. No. RPN2132) was used.

After detection, blocking was performed again and 1000 times diluted anti-synoviolin antibodies, and 5000 times diluted anti-α-tubulin antibodies (SIGMA Clone B-5-1-2), were used as the primary antibodies and incubated for 30 minutes. Subsequent incubation was carried out for 30 minutes using 10000 times diluted HRP-bound anti-mouse IgG (Amersham Biosciences, Cat. No. NA931V) as secondary antibodies. For detection, an ECL plus Western Blotting Detection System (Amersham Biosciences, Cat. No. RPN2132) was used.

As a result, the expression of both proteins could not be found due to the inhibition of the expression of SEL1L/hsHRD3 and Synoviolin (FIG. 11). That is, both proteins were found to be stabilized by each other.

### EXAMPLE 6

Effects of Stabilization of SEL1L/hsHRD3 and Complex Formation with Synoviolin

HEK293 cells were transfected with SP-HA-hsHRD3B and vector, or FLAG-Synoviolin plasmid. After 36 hours, cycloheximide was added to start the Chase Assay. The cells were recovered at 0, 1, 2, 4 and 6 hours and the total extraction solution was prepared. Each protein was detected by the Western blotting and analyzed quantitatively.

That is, HEK293 cells were seeded on a 6-well plate at 2×10⁵ cells. After 24 hours, the following two kinds of plasmid combinations, (g) and (h), were used for transfection.

(g) 0.5 μg of SP-HA-hsHRD3B/pcDNA3 and 0.25 μg of pcDNA3 vector

(h) 0.5 μg of SP-HA-hsHRD3B/pcDNA3 and 0.25 of FLAG-Synoviolin/pcDNA3

After 36 hours of transfection, the culture medium was replaced with a fresh medium. After an additional 2 hours, cycloheximide was added to have a final concentration of 30 μg/ml. At 0, 1, 2, 4, and 6 hours after, the cells were recovered and dissolved in a 50 μl of extraction buffer III (10 mM Tris-HCl pH 7.5, 5 mM EDTA, 1% NONIDET P-40, 0.1% SDS, 200 mM NaCl 10 mM NEM, 1 mM PMSF, 1 mM dithiothreitol, 0.1% Aprotinin, 0.5 μg/ml Pepstatin A, 1 μg/ml Leupeptin) and left on ice for 30 minutes. After centrifugation at 14000 rpm at 4° C. for 30 minutes, a 1 μl of supernatant was analyzed for the protein concentration using a Bio-Rad DC Protein Assay Reagent (BIO-RAD, Cat. No. 500-0116). A 15 μl of 4× SDS buffer was added to the remaining 45 μl, and the mixture was heated at 37° C. for 10 minutes. The cell-extracted solution in an equivalent of 10 μg was separated by electrophoresis using 7.5% acrylamide gel. After blotting on a nitrocellulose membrane (OPTITRAN BA-S 85, REINFORCED NC, Schleicher & Schuell, Cat. No. 10 439196), the membrane was blocked with 5% skim milk overnight. Incubation was carried out for 30 minutes using 10000 times diluted anti-HA antibodies (3F10, Roche, Cat. No. 1 867 431) as the primary antibodies, and followed by incubation for 30 minutes using 10000 diluted HRP-bound anti-rat IgG. For detection, an ECL plus Western Blotting Detection System (Amersham Biosciences, Cat. No. RPN2132) was used. The bands detected were quantitatively analyzed by the ImageJSoftware. For accurate measurement, sample at time 0 was diluted by 2 times and 4 times to draw a standard curve. Based on the standard curve, the ratios of the two parameters were estimated.

As a result, in the absence of Synoviolin, the half-life of SEL1L/hsHRD3 was reduced from 4.3 hours to 1.8 hours (FIGS. 12A, and 12B). That is, unless SEL1L/hsHRD3 forms a complex with Synoviolin, it was determined to be destabilized within the cells.

### INDUSTRIAL APPLICABILITY

The present invention provides a pharmaceutical composition containing a substance which inhibits hyperplasia in synovial cells (including synovial tissue) and the interleukin-6 production. Since this substance can inhibit hyperplasia in synovial tissue or in synovial cells, this invention is useful as a pharmaceutical composition for diagnosing and treating at least one disease selected from the group consisting of rheumatoid arthritis, fibrosis, arthritis, cancers, and cerebral neural diseases.

SEQUENCE LISTING FREE TEXT

SEQ ID NO 4: DNA/RNA bonded molecule
SEQ ID NO 5: DNA/RNA bonded molecule
SEQ ID NO 6: DNA/RNA bonded molecule
SEQ ID NO 7: DNA/RNA bonded molecule
SEQ ID NO 8: DNA/RNA bonded molecule
SEQ ID NO 9: DNA/RNA bonded molecule
SEQ ID NO 10: Synthesized DNA
SEQ ID NO 11: Synthesized DNA
SEQ ID NO 12: Synthesized DNA
SEQ ID NO 13: Synthesized DNA
SEQ ID NO 14: Synthesized DNA
SEQ ID NO 15: Synthesized DNA

1. A pharmaceutical composition containing a substance which inhibits the proliferation of synovial cells.

2. The pharmaceutical composition according to claim 1, wherein the substance which inhibits the proliferation of synovial cells is an expression-inhibitory substance for Synoviolin.

3. The pharmaceutical composition according to claim 2, wherein the expression-inhibitory substance for Synoviolin is a substance which inhibits the expression of a gene encoding hsHRD3.

4. The pharmaceutical composition according to claim 3, wherein the substance which inhibits the expression of a gene encoding hsHRD3 is a siRNA or shRNA against the gene encoding hsHRD3.

5. The pharmaceutical composition according to claim 3 or 4, wherein the gene encoding hsHRD3 contains a DNA selected from the following (a) or (b):
(a) A DNA consisting of the nucleotide sequence as shown in SEQ ID NO: 1;
(b) A DNA which hybridizes to a DNA complementary to a DNA consisting of the nucleotide sequence as shown in SEQ ID NO: 1 under stringent conditions, and encodes a protein having hsHRD3 activity.

6. The pharmaceutical composition according to claim 4, wherein the siRNA targets a portion of the nucleotide sequence as shown in SEQ ID NO: 1.

7. (canceled)

8. A method of inhibiting the proliferation of synovial cells, comprising inhibiting the expression of hsHDR3 in synovial cells.

9. A method of inducing apoptosis in synovial cells, cancer cells, leukemia or malignant tumors, comprising inhibiting the expression of hsHRD3 in the synovial cells.

10. A method of inhibiting collagen production in synovial cells, pulmonary fibrosis or hepatocirrhosis, comprising inhibiting the expression of hsHRD3 in synovial cells.

11. A method of inhibiting interleukin-6 production in at least one cell selected from the group consisting of synovial cells, cancer cells, leukemia cells, osteosarcoma cells, malignant tumor cells, immune system cells and osteoclast cells, comprising inhibiting the expression of hsHRD3 in synovial cells.

12. The method according to any one of claims 8 to 11, wherein the expression of hsHRD3 is inhibited by binding inhibition between hsHRD3 and Synoviolin.

13. A pharmaceutical composition comprising a substance which inhibits interleukin-6 production.

14. The pharmaceutical composition according to claim 13, wherein the substance which inhibits interleukin-6 production is an expression-inhibitory substance for Synoviolin.

15. The pharmaceutical composition according to claim 14, wherein the expression-inhibitory substance for Synoviolin is a substance which inhibits the expression of a gene encoding hsHRD3.

16. The pharmaceutical composition according to claim 15, wherein the substance which inhibits the expression of a gene encoding hsHRD3 is an siRNA or shRNA against the gene encoding hsHRD3.

17. The pharmaceutical composition according to claim 15 or 16, wherein the gene encoding hsHRD3 comprises a DNA selected from the following (a) or (b):
(a) A DNA consisting of the nucleotide sequence as shown in SEQ ID NO: 1;
(b) A DNA which hybridizes to a DNA complementary to a DNA consisting of the nucleotide sequence as shown in SEQ ID NO: 1 under stringent conditions, and encodes a protein having hsHRD3 activity.

18. The pharmaceutical composition according to claim 16, wherein the siRNA targets a portion of the nucleotide sequence as shown in SEQ ID NO: 1.

19. (canceled)

20. (canceled)

21. A method for treating a disease attributed to cellular hyperplasia, comprising administering to a subject in need thereof an effective amount of the pharmaceutical composition of any one of claims 1 to 4.

22. The method of claim 21, wherein said disease is at least one disease selected from the group consisting of rheumatoid arthritis, fibrosis, arthritis, cancers, and cerebral neural diseases.

23. A method for treating a disease associated with interleukin-6 production, comprising administering to a subject in need thereof an effective amount of the pharmaceutical composition of any one of claims 13 to 16.

24. The method of claim 23, wherein said disease is at least one disease selected from the group consisting of rheumatoid arthritis, multiple myeloma, Castleman's disease, Crohn's disease, systemic juvenile idiopathic arthritis, systemic lupus erythematosus, and osteoporosis.

25. A method for inhibiting an inflammatory reaction in a subject, comprising administering to the subject an effective amount of the pharmaceutical composition of any one of claims 13 to 16.
